(19) United States
(12) Patent Application Publication
(10) Pub. No.: US 2022/0236298 A1
(43) Pub. Date: Jul. 28, 2022

(54) INTERFACE FOR THE SYSTEM OF SAMPLING, PREPARATION, AND ANALYSIS OF A SAMPLE, ESPECIALLY BY CAPILLARY ELECTROPHORESIS

(71) Applicant: Univerzita Karlova, Prague (CZ)

(72) Inventor: Petr TUMA, Divci Kopy (CZ)

(21) Appl. No.: 17/596,171

(22) PCT Filed: Jun. 5, 2020
(86) PCT No.: PCT/CZ2020/050042

§ 371 Date: Dec. 3, 2021

(30) Foreign Application Priority Data
Jun. 5, 2019 (CZ) PV 2019-350

(51) Int. Cl. G01N 35/10; G01N 27/447; A61B 5/145

(52) U.S. Cl. CPC G01N 35/1095; G01N 27/44743; A61B 2562/125; G01N 2035/1034; A61B 2562/028; A61B 5/14546

(57) ABSTRACT

The interface for the system of sampling, preparation, and analysis of a sample, especially by capillary electrophoresis, consists of a cross connector, sampling capillary, separation capillary, capillary supplying BGE, and ground capillary, wherein walls of the cross connector channels fit tightly onto at least three capillaries of an outer diameter of 300 μm to 800 μm, and the internal volume of the cross connector is less than 0.5 μL, and the capillary, onto which the cross connector channel does not fit, is sealed in the cross connector channel with an adhesive.

### TECHNICAL FIELD

The invention falls under the field of devices for medical diagnostics and analysis of biological material.

### BACKGROUND ART

Microdialysis (MD) is a modern technique for sampling living tissues and organs in order to monitor biochemical and physiological processes at the molecular level. The basis of the microdialysis sampling is an implementation of a miniature microdialysis probe into the studied object, such as living tissue, organ or even a completely different type of complex sample. The actual implementation of the probe is highly local and, at the same time, minimally invasive, so the influence on the monitored processes is low. The microdialysis probe is equipped with a selective membrane, one side of which is in direct contact with the studied object and the other side is washed with an acceptor solution, which is most often represented by saline in the case of human and animal tissues and organs. During microdialysis, substances are selectively transferred from the living tissue through the membrane into the saline, which is referred to as microdialysate after enriching. The supply of saline and the discharge of the obtained microdialysate are provided by thin tubings. A high yield of microdialysis collection can be ensured only at a low flow rate of the microdialysate through the probe, ranging 0.5-10 μL/min, and is controlled by a linear pump with a syringe inserted. A maximum of 10-50 μL of microdialysate is obtained by the procedure described, placing specific requirements for subsequent analysis.

A suitable microanalytical technique capable of reproducibly analysing microliter volumes of sample is represented by capillary electrophoresis (CE) performed in capillaries of inner diameter (ID) of 10-50 μm and outer diameter (OD) of about 380 μm. The volume of the sample dosed into the capillary is only in submicroliter amounts, which provides a room for repeated analyses of the microdialysate. In addition, CE is characterized by high separation efficiency, which is a necessary condition for the determination of one specific metabolite or a group of structurally similar substances side by side in complex clinical matrices. Also, the separation time in CE is short and usually does not exceed 3-5 minutes, providing the possibility to perform rapid sequence analyses and monitor metabolite levels over time. Commonly available CE instruments are adapted for off-line analysis of microdialysates, which is performed in a standard way. The collected microdialysates are usually frozen firstly and stored for a long time; prior to the CE analysis, the thawed samples are processed by laboratory procedures, such as filtration, dilution, derivatization, extraction, centrifugation, etc.; and subsequently, the treated sample is subjected to the instrumental analysis.

Significantly more practical is the on-line CE analysis of the collected microdialysate flowing out of the probe and real-time monitoring of biochemical and physiological processes so that the sample does not have to be manually treated; its consumption for analysis is significantly lower and the sequential analysis performing is allowed in shorter time intervals. However, commonly available electrophoretic devices are not adapted for these purposes.

The solution is to connect the output of the microdialysis and the inlet to the electrophoretic device using a so-called flow-gating interface (FGI), at which the microdialysate flow is diverted from the inlet to the separation capillary by a carrier solution stream usually represented by the background electrolyte (BGE). Only at the moment of dosing, the BGE stream is stopped for a certain time, causing accumulation of the microdialysate in the space before entering the separation capillary, at this moment the microdialysate is dosed into the capillary, then the microdialysate flow is diverted from the entrance to the separation capillary again, and the CE separation is started.

In practice, several types of FGI are used, which differ from each other in structure and principle of operation. For example, IN VIVO MONITORING OF GLUTATHIONE AND CYSTEINE IN RAT CAUDATE NUCLEUS USING MICRODIALYSIS ON-LINE WITH CAPILLARY ZONE ELECTROPHORESIS-LASER INDUCED FLUORESCENCE DETECTION (Lada, Kennedy; 1997) describes an FGI formed by a reaction capillary, where biological sample derivatization occurs and which is fixed in a block of polycarbonate together with the separation capillary. The two capillaries are separated by a channel drilled in the block. In this case, it is a highly specific device with different FGI parameters, suitable only for monitoring sulphur compounds and is used in conjunction with laser-induced fluorescence, not electrophoresis.

Furthermore, AN AIR-ASSISTED FLOW-GATING INTERFACE FOR CAPILLARY ELECTROPHORESIS (Opekar, Tůma; 2019) describes an FGI where sampling and separation capillaries are placed in a shared tube, which is a part of channel system and an air stream is used to purge the tube. Moreover, this described FGI was not used in connection with microdialysis in this example.

The document HYDRODYNAMIC SAMPLE INJECTION INTO SHORT ELECTROPHORETIC CAPILLARY IN SYSTEMS WITH A FLOW-GATING INTERFACE (Opekar, Tůma; 2017) presents the use of FGI in the form of a cross connector formed by a square piece of Plexiglass with drilled channels of diameter of 1.55 mm into which four capillaries are placed—sampling capillary, separation capillary, capillary feeding BGE, and waste capillary. Although this solution is simpler and more user-friendly than the previous two mentioned, it still has a number of disadvantages. The channels are significantly wider than the capillaries placed in them, and, therefore, the connector has a large internal (so-called dead) volume of approximately 50 μL and at least 3 μL, resulting in mixing the sample with the surrounding electrolyte and in time-consuming and incomplete washing out of the electrolyte. As a result, this method is only suitable for industrial purposes where a large sample volume is available, not for monitoring metabolites in living subjects. In addition, due to incomplete washing out electrolyte, a significant measurement error may occur. Due to the washing-out time of the electrolyte, which is directly proportional to the size of the internal volume, in this case, 4 s to 8 s, the time resolution in the sequence analysis is significantly limited. Adjusting the capillaries in the channels in order to sample effectively, although with the limitations mentioned above, is also very demanding and requires the use of special adhesive or Teflon tubes to seal the channels.

Therefore, the object of the present invention is to provide an interface having very small internal volume and allowing its use for microdialysis applications providing a very small amount of sample, for example, the analysis of samples obtained directly from living subjects.

### SUMMARY OF INVENTION

The present invention provides a cross connector for use as an FGI in the electrophoretic analysis of samples obtained by microdialysis. The connector is formed by a block of material allowing the formation of a hydrodynamically tight connection to the connected capillaries. Inside the connector, four capillaries are arranged in the channels so that the capillaries fit tightly onto the walls of the channels to fill them completely. The capillary means any cylindrical component with a continuous cavity of outer diameter less than or equal to 800 μm.

Due to the fact that the walls of the channels fit tightly onto the capillaries, it is not necessary to adjust the position of the capillaries and seal the channels with adhesive before measuring. The component is self-sealing. Hydrodynamically tight connection in such connector can be achieved by using capillaries of outer diameter of at least 300 μm. When using thinner capillaries, it is necessary to fix these capillaries, for example, with an adhesive. However, the effect of the invention is not achieved in this form, so it is necessary that at least three of the four capillaries have an outer diameter equal to or higher than 300 μm to achieve hydrodynamically tight connection.

The four capillaries, which are the sampling capillary, the separation capillary, the capillary supplying BGE, and the waste capillary, are arranged at the cross connector in a cross shape so that adjacent capillaries always form an angle of 90° to each other and there is no free space that would contribute to increasing of the internal volume along at least three capillaries, onto which the cross connector channels fit tightly. This assembly provides an internal, so-called dead, connector volume of less than 0.5 μL. Due to the small internal volume, after pausing the BGE flow, the BGE is rapidly washed out and replaced by a sample, which significantly reduces the probability of measurement error due to the presence of residual electrolyte compared to the state of art. The smallest possible internal volume is therefore essential, as it makes it possible to use this method to combine capillary electrophoresis with microdialysis applications providing a very small amount of sample, for example by analysing samples obtained directly from living subjects.

### BRIEF DESCRIPTION OF DRAWINGS

FIG. 1 depicts the connection diagram of the interface of Example 1.

FIG. 2 depicts a detail of the cross connector connected in the interface of Example 1.

FIG. 3 depicts the analysis result achieved according to Example 2, while EOF signifies electroosmotic flow, Suc signifies sucrose, Lac signifies lactose, Gal signifies galactose, Glc signifies glucose, Fru signifies fructose.

FIG. 4 depicts the analysis result achieved according to Example 3, while Lys signifies lysine, Arg signifies arginine, His signifies histidine, Gly signifies glycine, Ala signifies alanine, Val signifies valine, Ile signifies isoleucine, Leu signifies leucine, Ser signifies serine, Thr signifies threonine, Gln signifies glutamine, Glu signifies glutamic acid, Tyr signifies tyrosine, Asp signifies aspartic acid.

FIG. 5 depicts the analysis result achieved according to Example 4, while Lys signifies lysine, Arg signifies arginine, His signifies histidine, Gly signifies glycine, Ala signifies alanine, Val signifies valine, Ile signifies isoleucine, Leu signifies leucine, Ser signifies serine, Thr signifies threonine, Met signifies methionine, Gln signifies glutamine, Tyr signifies tyrosine, Asp signifies aspartic acid.

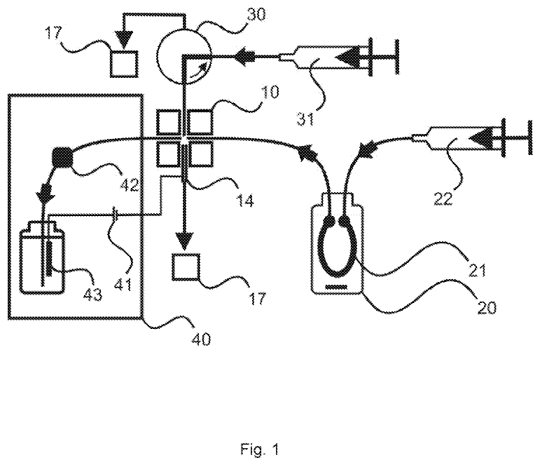

### DESCRIPTION OF EMBODIMENTS

### Example 1

Example 1 demonstrates a laboratory-validated interface connection between the microdialysis probe (21) and the analytical instrument (40), in this example an electrophoretic instrument.

1) The capillary (11) of ID/OD of 100/360 μm, length of 3.0 cm, is connected to the upper inlet of the cross connector (10), at the other end, the capillary (11) is connected to the outlet of the three-way valve (30). Through this upper inlet, the carrier solution is fed to the cross connector (10) at a flow rate of 83 μL/min; the carrier solution is pumped from the 50-mL filling vessel (31), for example, a syringe, using a linear pump. When switching the valve (30) to the closed position during dosing of the sample into the analytical device (40), the carrier solution is discharged from the valve (30) by a side path directly into the waste (17).

2) The microdialysate is introduced into the right inlet of the cross connector (10) via the capillary (12) of ID/OD of 100/150 μm; the tightness of the connection of a thinner capillary in the cross connector (10) is ensured by the UV-curable adhesive (13), which is applied around the outer surface of the capillary, 2 mm in front of its orifice. The microdialysis probe (21) itself is laboratory-made of 4.0 cm long microdialysis tubing of ID/OD of 200/216 μm, cut-off 13 kDa, into both ends of which two silica capillaries of ID/OD 75/150 μm are inserted to a depth of 1 mm, and the UV-curable adhesive is applied on the connections. The inlet capillary is connected to the 5-mL filling vessel (22) with an acceptor solution, the solution flow of 5 μL/min is ensured by a linear pump. The microdialysis probe (21) is immersed in the sample (20) and the microdialysate is fed through the outlet capillary to the cross connector (10).

3) The 15-mm ground, preferably stainless steel, capillary (14), for example, made from an injection needle of ID/OD of 400/800 μm, is inserted into the lower inlet of the cross connector (10); and a grounding contact from the high-voltage source (41) is connected to the ground capillary (14). The other end of the capillary (14) is connected to the 5 cm long capillary of ID/OD of 100/360 μm using the UV-curable adhesive; this capillary discharges the solution from the cross connector (10) to the waste (17).

4) The dosing end of the separation capillary (15) is inserted into the left inlet of the cross connector (10), and this end is led out of the analytical instrument (40).

5) The cross connector (10) connected in this way has its internal (dead) volume (16) of approximately 0.1 μL.

In this case, the interface is connected to a commercially available electrophoretic instrument. The dosing end of the separation capillary (15) is pulled out of the instrument and connected to the cross connector (10). The other end of the separation capillary (15) remains in the analytical instrument (40) and is inserted into the end electrophoretic vial which is connected to a pressure pump; from this end, the separation capillary is washed with BGE between the individual analyses by applying an overpressure of 920 mbar or, conversely, a sample is dosed into its dosing end by applying a vacuum of −50 mbar. The high-voltage electrode (43) is also located in the end vial in the analytical instrument (40), and the ground electrical contact is led out of the instrument using an insulated conductor and connected to the stainless-steel capillary (14). Thus, the microdialysis moiety of the apparatus is at zero ground potential and there is no risk of injury.

The entire analysis process, including washing the capillary, dosing the sample (20), applying the separation voltage, controlling the experiment, including collecting and processing the data, is performed by a computer programme. Synchronization of the analysis with the external valve (30) is ensured using an A/D converter, which simultaneously serves to collect the analogue signal from the non-contact conductivity detector (42), which is built into the electrophoretic cassette of the analytical instrument (40), where it is thermostated.

### Example 2

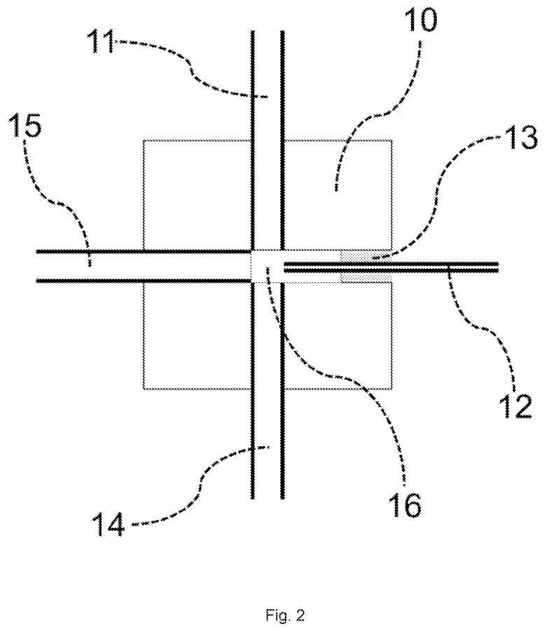

Example 2 demonstrates the interface connection parameters according to Example 1 to determine the carbohydrate profile in blueberry-flavoured fruit yoghurt, where the laboratory-made microdialysis probe (21) was inserted directly into untreated yoghurt and washed with 0.01 M NaOH acceptor solution, wherein the results of the analysis are shown in FIG. 2.

CE-MD determination of carbohydrates in blueberry-flavoured fruit yoghurt CE: capillary, ID/OD: 10/360 μm, length/length to detector: 35/22 cm; BGE, 50 mM NaOH, pH 12.6; separation voltage/current +10 kV/3 μA, hydrodynamic dosing with vacuum −50 mbar for 1 s; microdialysis: acceptor solution—0.01 M NaOH, flow 10 μL/min, laboratory-made microdialysis probe—length: 40 mm, OD: 216 μm, cut-off 13 kDa; the probe directly immersed in the yoghurt; FGI: carrier solution—BGE, flow 83 μL/min.

### Example 3

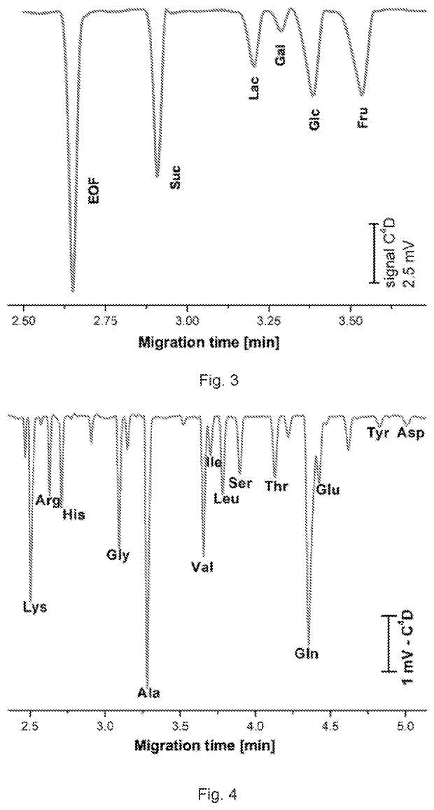

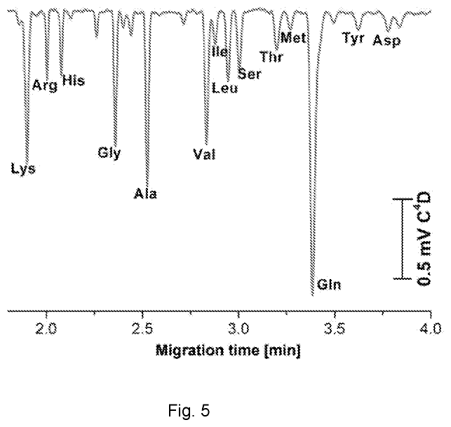

The Example 3 demonstrates interface connection according to Example 1 for in-vitro determination of amino acid profile in human blood plasma, where a microdialysis probe intended for clinical use was directly implemented into a sample of untreated human blood plasma placed in a test tube. The microdialysis probe (21) was washed with saline, and the obtained microdialysate was analysed to determine the profile of free plasma amino acids; the results of the analysis are shown in FIG. 3.

CE-MD determination of amino acids in human blood plasma. CE: capillary, ID/OD: 25/360 μm, length/length to detector: 36/23 cm; BGE, 3.2 M acetic acid +20% v/v methanol, pH 2.1; separation voltage/current +30 kV/3.5 μA, hydrodynamic dosing with vacuum −50 mbar for 20 s; microdialysis: acceptor solution—saline, flow 2 μL/min, clinical microdialysis probe—length/diameter: 20/0.5 mm, cut-off 20 kDa; the probe directly immersed in blood plasma; FGI: carrier solution—BGE, flow 83 μL/min.

### Example 4

Example 4 demonstrates interface connection parameters according to Example 1 for in-vivo determination of amino acid profile in human adipose tissue, where a microdialysis probe intended for clinical use was directly implemented into the subcutaneous tissue of human abdominal adipose tissue. The microdialysis probe (21) was washed with saline, and the obtained microdialysate was analysed to determine the profile of amino acids; the results of the analysis are shown in FIG. 4.

CE-MD real-time monitoring of the amino acid profile in human abdominal adipose tissue. CE: capillary, ID/OD: 25/360 μm, length/length to detector: 36/23 cm; BGE, 3.2 M acetic acid +20% v/v methanol, pH 2.1; separation voltage/current +30 kV/3.5 μA, hydrodynamic dosing with vacuum −50 mbar for 10 s; microdialysis: acceptor solution—saline, flow 2μL/min, clinical microdialysis probe—length/diameter 30/0.5 mm, cut-off 20 kDa; the probe directly implemented in adipose tissue; FGI: carrier solution—BGE, flow 83 μL/min.

### INDUSTRIAL APPLICABILITY

The interface for the system of sampling, preparation and analysis of a sample, especially by capillary electrophoresis, is industrially applicable in medicine for the analysis of samples obtained from a human body as well as in industrial production for the analysis of biological, food and environmental materials.

1. The interface for the system of sampling, preparation, and analysis of a sample by capillary electrophoresis, the interface comprising:
a cross connector,
sampling capillary,
separation capillary,
capillary supplying BGE, and
ground capillary,
wherein walls of the cross connector channels fit tightly onto at least three capillaries of outer diameter of 300 μm to 800 μm, and the internal volume of the cross connector is less than 0.5 μL.

2. The interface according to claim 1, wherein the internal volume of the cross connector is 0.1 μL.

3. The interface according to claim 1, wherein the capillary, onto which the cross connector channel does not fit, is sealed in the cross connector channel with an adhesive.

4. The interface according to claim 1, wherein the capillary, onto which the cross connector channel does not fit, is sealed in the cross connector channel with an adhesive.
